(19) United States
(12) Patent Application Publication
(10) Pub. No.: US 2021/0187157 A1
(43) Pub. Date: Jun. 24, 2021

(54) BIODEGRADABLE IRON-BASED ALLOY COMPOSITION, MEDICAL IMPLANT APPLYING THE SAME, AND MANUFACTURING METHOD THEREOF

(71) Applicant: INDUSTRIAL TECHNOLOGY RESEARCH INSTITUTE, Hsinchu (TW)

(72) Inventors: Tai-Sheng CHEN, Zhudong Township (TW); Hong-Jen LAI, Hsinchu City (TW); Ming-Sheng LEU, Zhudong Town (TW); Wei-Chin HUANG, Tainan City (TW); I-Chun LAI, Tainan City (TW)

(73) Assignee: INDUSTRIAL TECHNOLOGY RESEARCH INSTITUTE, Hsinchu (TW)

(21) Appl. No.: 17/127,171

(22) Filed: Dec. 18, 2020

Related U.S. Application Data
(60) Provisional application No. 62/953,643, filed on Dec. 26, 2019.

(30) Foreign Application Priority Data
Dec. 19, 2019 (TW) 108146644

(51) Int. Cl. A61L 27/04; A61L 27/58

(52) U.S. Cl. CPC A61L 27/042; B33Y 10/00; A61L 27/58

(57) ABSTRACT

A biodegradable iron-based alloy composition, a medical implant applying the iron-based alloy composition, and a manufacturing method of the medical implant are provided. The biodegradable iron-based alloy composition includes at least 98 wt % of iron and 2 wt % or less of an additional material. The additional material includes 0.1 wt %-0.8 wt % of Mn, 0.01 wt %-0.15 wt % of Mo, 0.1 wt %-0.3 wt % of Cr, 0.02 wt %-0.15 wt % of C, and 0.01 wt %-0.15 wt % of Si.

### CROSS REFERENCE TO RELATED APPLICATIONS

This application claims the benefit of U.S. Provisional Application No. 62/953,643, filed on Dec. 26, 2019, which is hereby incorporated herein by reference.

The application is based on, and claims priority from, Taiwan Application Serial Number 108146644, filed on Dec. 19, 2019, the disclosure of which is hereby incorporated by reference herein in its entirety.

### TECHNICAL FIELD

The disclosure relates in general to a biodegradable composition, a medical implant applying the composition, and a manufacturing method thereof, and in particular it relates to a biodegradable iron-based alloy composition, a medical implant applying the iron-based alloy composition, and a method of manufacturing the medical implant applying the same.

### BACKGROUND

A graying society is an irreversible global trend. This is the increase in the percentage of older individuals in a society due to fertility declines, rising life expectancy, and population aging. As a result, medical expenses will grow continuously and the demand for medical implants will also increase. Typical medical implants, such as bone nails and bone plates used in orthopedic surgeries, are made of metal (such as stainless steel, cobalt chromium alloy, titanium, and titanium alloy) and have high strength, high toughness, high fatigue resistance, high corrosion resistance, high plasticity, high workability, and affordability. While a metal medical implant does not degrade after being implanted in a human body, it does carry the potential risk of infection, however. Normally, after a wound heals, a second surgery is required to remove the metal medical implant from the body. It is this second surgery, for removing the medical implant, that has the clinical risk. According to current statistical analysis, about 12%-40% of people suffer from complications, and most of the complications are nerve damage. Also, the local space of tissue may be compressed after recovery from an ossium fracture, and it may further ulcerate the epidermis and soft tissue.

The second surgery for removing the medical implant has the clinical risk of causing complications and damaging nerves. Therefore, a new technology for fabricating a medical implant using biodegradable polymers such as polylactic acid (PLA), polyglycolic acid (PGA), polycyanoacrylate (PACA) is provided in response to this need. The medical implant formed of polymers can be absorbed by the human body, and there is no need to perform the second surgery to remove it from the body, hence avoiding extra risks and harm to the patient. However, the medical implant formed of biodegradable polymer materials still has problems: it is lacking sufficient mechanical strength, it has poor mechanical properties and a quick degradation rate, and it is unable to bear excessive stress, which may have adverse effects on the human body.

Therefore, it has become an important task for the medical industry to develop an advanced metal biodegradable composition, a medical implant applying the composition, and a method of manufacturing the same.

### SUMMARY

The disclosure relates to a biodegradable iron-based alloy composition, a medical implant applying the iron-based alloy composition, and a method of manufacturing the medical implant applying the same. According to embodiments of the present disclosure, a biodegradable iron-based alloy composition includes at least 98 wt % of iron (Fe) and 2 wt % or less of an additional material. Therefore, a biodegradable medical implant made of a biodegradable iron-based alloy composition has advantages such as non-cytotoxicity and high biocompatibility. Also, the additional material of the iron-based alloy composition greatly improves the mechanical strength and the degradation rate, which reduces or avoids the risk of medical implant breaking in the body.

Some embodiments of the present disclosure provide a biodegradable iron-based alloy composition including at least 98 wt % of iron (Fe) and 2 wt % or less of an additional material, wherein the additional material includes 0.1 wt %-0.8 wt % of manganese (Mn), 0.01 wt %-0.15 wt % of molybdenum (Mo), 0.1 wt %-0.3 wt % of chromium (Cr), 0.02 wt %-0.15 wt % of carbon (C), and 0.01 wt %-0.15 wt % of silicon (Si).

Some embodiments of the present disclosure provide a biodegradable medical implant. The biodegradable medical implant is made of the aforementioned biodegradable iron-based alloy composition.

Some embodiments of the present disclosure provide a method of manufacturing a biodegradable medical implant. The method includes providing a biodegradable iron-based alloy composition. The biodegradable iron-based alloy composition includes at least 98 wt % of iron (Fe) and 2 wt % or less of an additional material, wherein the additional material includes 0.1 wt %-0.8 wt % of manganese (Mn), 0.01 wt %-0.15 wt % of molybdenum (Mo), 0.1 wt %-0.3 wt % of chromium (Cr), 0.02 wt %-0.15 wt % of carbon (C), and 0.01 wt %-0.15 wt % of silicon (Si). The method further includes performing an additive manufacturing process on the biodegradable iron-based alloy composition to form the biodegradable medical implant.

A detailed description is given in the following embodiments.

### DETAILED DESCRIPTION

According to embodiments of the present disclosure, a biodegradable iron-based alloy composition including at least 98 wt % of iron (Fe) and 2 wt % or less of an additional material is provided. A biodegradable medical implant made of a biodegradable iron-based alloy composition has advantages of non-cytotoxicity and high biocompatibility. Also, the additional material of the iron-based alloy composition greatly improves the mechanical strength and the degradation rate, which reduces or avoids the risk of medical implant breaking in the body. Some embodiments are described below. It should be understood that the compositions specified in the embodiments are provided for illustration, not for limiting the scope of the present disclosure. It is understood by people having ordinary skill in the art that the compositions can be modified or changed according to the requirements in practical application.

It is to be understood that the terminology used herein is used for the purpose of describing particular embodiments only, and is not intended to limit the scope of the present disclosure. It must be noted that as used herein and in the appended claims, the singular forms “a,” “an,” and “the” include the plural reference unless the context clearly dictates otherwise. It is to be further understood that the words “comprising” and/or “including” herein are used to indicate the steps, operations, components and/or properties in the descriptions, but those words are not used to exclude one or more additional steps, operations, components, properties, and a combination thereof.

Moreover, the phrase “one of the embodiments” or “an embodiment” means that the specific steps, operations, components and/or properties described in the context are included in at least one embodiment. Thus, the phrase “according to one of the embodiments” or “in one embodiment” throughout the specification are not necessarily referring to the same embodiment. Also, certain steps, operations, components and/or properties many be combined in any suitable manner in one or more embodiments. It can be understood that additional steps can be provided before, during and after the method, and some of the described steps can be replaced or eliminated in other embodiments of the method.

Unless defined otherwise, all terms (including technical and scientific terms) used herein have the same meanings as commonly understood by one of ordinary skill in the art to which this disclosure belongs. It should be appreciated that, in each case, the term, which is defined in a commonly used dictionary, should be interpreted as having a meaning consistent with the context of related art and this disclosure, and should not be interpreted as in an idealized or overly formal manner, unless the term is specifically defined.

Also, the pure element mentioned in the full context of the present disclosure, such as pure iron, refers to an element free of impurities such as other elements and compounds. However, it is difficult to completely remove the impurities in the processes such as the smelting, refining, coating processes to achieve a mathematically or theoretically 100% pure metal. Therefore, when an amount of impurity of a composition is in an allowable range of an element as defined by the corresponding standards or specifications, it can be regarded as “pure element”. It is understood by one of ordinary skill in the art that the corresponding standards or specifications for defining an “pure element” would be different according to different properties, conditions, requirements, etc.; therefore, specific standards or specifications are not listed in the context below.

According to embodiments of the present disclosure, a biodegradable iron-based alloy composition is provided. The biodegradable iron-based alloy composition includes at least 98 wt % of iron (Fe) and 2 wt % or less of an additional material. The additional material includes 0.1 wt %-0.8 wt % of manganese (Mn), 0.01 wt %-0.15 wt % of molybdenum (Mo), 0.1 wt %-0.3 wt % of chromium (Cr), 0.02 wt %-0.15 wt % of carbon (C), and 0.01 wt %-0.15 wt % of silicon (Si).

Typically, a biodegradable magnesium (Mg)-based composition is used to fabricate a medical implant. However, the degradation rate of the medical implant made of the biodegradable magnesium-based composition in the human body is very fast, resulting in insufficiency of mechanical rigidity of the medical implant and difficulty of maintaining mechanical strength for human body. Also, hydrogen generated during the degradation of the magnesium-based composition may increase the risk of inflammation in the body. In addition, the medical implant made of the magnesium-based composition often contains trace amounts of rare earth elements, and this has raised concerns for neurotoxicity. On the other hand, a medical implant made of pure iron-based biodegradable compositions has a relatively slow degradation rate, which makes an existing time in human body difficult to be adjusted and control.

According to embodiments of the present disclosure, a biodegradable iron-based alloy composition includes at least 98 wt % of iron (Fe) and 2 wt % or less of an additional material. Thus, a medical implant made of the biodegradable iron-based alloy composition has advantages of not only non-toxicity to cells but also high biocompatibility. Also, the mechanical strength and the degradation rate of a medical implant made of the biodegradable iron-based alloy composition can be greatly increased by adding a small amount of the additional material, which reduces or avoids the risk of medical implant breaking in the body.

According to some embodiments of the present disclosure, the biodegradable iron-based alloy composition includes merely 0.1 wt %-0.8 wt % of manganese (Mn), which prevents biotoxicity caused by a high amount of manganese.

In some embodiments, the biodegradable iron-based alloy composition includes 98.2 wt %-99.7 wt % of iron (Fe) and 0.3 wt %-1.8 wt % of an additional material. In some embodiments, the biodegradable iron-based alloy composition includes 98.2 wt %-99.5 wt % of iron (Fe) and 0.5 wt %-1.8 wt % of an additional material. In some embodiments, the biodegradable iron-based alloy composition includes 98.2 wt %-99 wt % of iron (Fe) and 1 wt %-1.8 wt % of an additional material.

According to some embodiments of the present disclosure, the biodegradable iron-based alloy composition includes a certain large amount of iron (Fe) and a certain small amount of an additional material, as described above. Therefore, a medical implant made of the biodegradable iron-based alloy composition of the embodiments has advantages of excellent biocompatibility, sufficient biological degradation rate and high mechanical strength.

In some embodiments, the additional material of the biodegradable iron-based alloy composition includes 0.2 wt %-0.65 wt % of manganese (Mn) and 0.03 wt %-0.15 wt % of silicon (Si). In some embodiments, the additional material of the biodegradable iron-based alloy composition includes 0.3 wt %-0.6 wt % of manganese (Mn) and 0.06 wt %-0.12 wt % of silicon (Si).

According to some embodiments of the present disclosure, a certain amount of manganese (Mn) as described above in the biodegradable iron-based alloy composition can form a solid solution alloy with iron, which improves an overall strength of the iron-based alloy. In addition, the biodegradable iron-based alloy composition including a certain amount of silicon (Si) as described above can greatly improve material flow during subsequent processing and casting. Therefore, according to some embodiments of the present disclosure, the biodegradable iron-based alloy composition including certain amounts of manganese (Mn) and silicon (Si) as described above has advantages of improves an overall strength of the iron-based alloy and material flow, thereby improving the convenience of the processing procedures and increasing the mechanical strength of the product.

In some embodiments, the weight ratio of chromium to carbon (Cr/C) is in a range of about 0.6 to 10. In some embodiments, the weight ratio of chromium to carbon (Cr/C) is in a range of about 1 to 7. In some embodiments, the weight ratio of chromium to carbon (Cr/C) is in a range of about 1.3 to 5.

According to one of the biodegradable iron-based alloy compositions in some embodiments of the present disclosure, if the weight ratio of chromium to carbon (Cr/C) is in the certain range as described, this facilitates formation of the chromium carbide particles in an alloy structure after processing the biodegradable iron-based alloy composition. Chromium carbide has high hardness and wear/corrosion resistance. Therefore, mechanical strength of a medical implant made of the biodegradable iron-based alloy containing the chromium carbide particles of the embodiment can be greatly increased. In some embodiments, the chromium carbide particles may include Cr₃C₂, Cr₇C₃ and/or Cr₂₃C₆.

Moreover, according to one of the biodegradable iron-based alloy compositions in some embodiments of the present disclosure, chromium carbide particles can be formed after processing the biodegradable iron-based alloy compositions by an additive manufacturing process (also known as a 3D printing process), wherein the chromium carbide particles have relative small particle sizes. A medical implant made of the biodegradable iron-based alloy compositions containing these chromium carbide particles of the embodiment has a relative high mechanical strength. In one example, the chromium carbide particles have an average particle size about 10 and a medical implant as fabricated has a tensile strength of about 1200 MPa.

In some embodiments, the weight ratio of molybdenum to carbon (Mo/C) is in a range of about 0.06 to 6. In some embodiments, the weight ratio of molybdenum to carbon (Mo/C) is in a range of about 0.1 to 2. In some embodiments, the weight ratio of molybdenum to carbon (Mo/C) is in a range of about 0.5 to 0.8.

According to some embodiments of the present disclosure, if the weight ratio of molybdenum to carbon (Mo/C) is in the certain range as described, this facilitates formation of the molybdenum carbide particles in an alloy structure after processing the biodegradable iron-based alloy composition. Molybdenum carbide has high melting point, high hardness and high stability of mechanical strength. Therefore, mechanical strength of a medical implant made of the molybdenum carbide particles of the embodiment can be greatly increased. In some embodiments, the molybdenum carbide particles may include Mo₂C and/or MoC.

Moreover, according to one of the biodegradable iron-based alloy compositions in some embodiments of the present disclosure, molybdenum carbide particles can be formed by an additive manufacturing process (also known as a 3D printing process), thereby having relative small particle sizes. A medical implant made of the biodegradable iron-based alloy compositions containing these molybdenum carbide particles of the embodiment has a relative high mechanical strength. In one example, the molybdenum carbide particles have an average particle size of about 10 μm, and a medical implant as fabricated has a tensile strength of about 1200 MPa.

In some embodiments, the additional material of the biodegradable iron-based alloy composition further includes 0.01 wt %-0.2 wt % of copper (Cu). In some embodiments, the additional material of the biodegradable iron-based alloy composition further includes 0.05 wt %-0.15 wt % of copper (Cu). In some embodiments, the additional material of the biodegradable iron-based alloy composition further includes 0.08 wt %-0.14 wt % of copper (Cu).

According to some embodiments of the present disclosure, the biodegradable iron-based alloy composition may include a certain amounts of copper (Cu) as described above, so that a medical implant made of the biodegradable iron-based alloy composition of this embodiment has better corrosion resistance in the atmosphere. Therefore, the alloy structure of this embodiment would not embrittle easily and adversely affect the mechanical strength.

In some embodiments, the additional material of the biodegradable iron-based alloy composition includes 0.1 wt %-0.5 wt % of zinc (Zn). In some embodiments, the additional material of the biodegradable iron-based alloy composition includes 0.15 wt %-0.45 wt % of zinc (Zn). According to some embodiments of the present disclosure, the biodegradable iron-based alloy composition includes a certain large amount of zinc (Zn) as described above, thereby increasing a biological degradation rate of the biodegradable iron-based alloy composition.

In some embodiments, the additional material of the biodegradable iron-based alloy composition includes 0.01 wt %-0.1 wt % of phosphorum (P). In some embodiments, the additional material of the biodegradable iron-based alloy composition includes 0.02 wt %-0.06 wt % of phosphorum (P).

In some embodiments, the additional material of the biodegradable iron-based alloy composition includes 0.01 wt %-0.1 wt % of cobalt (Co). In some embodiments, the additional material of the biodegradable iron-based alloy composition includes 0.02 wt %-0.06 wt % of cobalt (Co).

Moreover, according to some embodiments of the present disclosure, the additional material of the biodegradable iron-based alloy composition does not include zirconium (Zr) or merely includes less than 0.6 wt % of zirconium (Zr), which prevents biotoxicity caused by a high amount of zirconium.

Moreover, according to some embodiments of the present disclosure, the additional material of the biodegradable iron-based alloy composition includes 0.02 wt % to 0.12 wt % of nickel (Ni). In some embodiments, the additional material of the biodegradable iron-based alloy composition includes 0.05 wt % to 0.1 wt % of nickel (Ni). In some other embodiments, the additional material of the biodegradable iron-based alloy composition does not include nickel (Ni). According to some embodiments of the present disclosure, the biodegradable iron-based alloy composition that includes a certain amount of nickel (Ni) as described above may increase the toughness and hardenability of the biodegradable iron-based alloy composition.

Moreover, according to some embodiments of the present disclosure, the additional material of the biodegradable iron-based alloy composition includes 0.01 wt % to 0.1 wt % of aluminum (Al). In some embodiments, the additional material of the biodegradable iron-based alloy composition includes 0.06 wt % to 0.09 wt % of aluminum (Al). In some other embodiments, the additional material of the biodegradable iron-based alloy composition does not include aluminum (Al). According to the aforementioned embodiments, the additional material(s) of the iron-based alloy composition can form smaller grains and greatly improve the mechanical strength of the biodegradable medical implant as made.

It is known that the degradation rate of a medical implant made of the magnesium (Mg)-based composition in the human body is very fast. Also, hydrogen generated during the degradation of the magnesium-based composition may increase the risk of inflammation in the body and may toxic to the nervous system. According to some embodiments of the present disclosure, the additional material of the biodegradable iron-based alloy composition does not include magnesium (Mg), which prevents biotoxicity caused by magnesium.

Moreover, according to some embodiments of the present disclosure, the biodegradable iron-based alloy composition may not substantially include a biodegradable polymer material, such as polylactic acid (PLA), polyglycolic acid (PGA), polyakyl cyanoacrylate (PACA), polycaprolactone (PCL), polydioxanone (PDO), or a combination thereof. Additionally, according to some embodiments of the present disclosure, the biodegradable iron-based alloy composition may not substantially include a biodegradable ceramic material, such as tricalcium phosphate (TCP), titanium oxide, aluminum oxide, silicon oxide, zirconia oxide, or a combination thereof.

According to some embodiments of the present disclosure, the biodegradable iron-based alloy composition includes iron (Fe) as the main component and trace amount of one or more additive materials as described above. Thus, the medical implant made of the biodegradable iron-based alloy composition of the embodiments has good biocompatibility, excellent mechanical strength and suitable degradation rate, without adding any biodegradable polymer material and/or any biodegradable ceramic material.

In some embodiments, the biodegradable iron-based alloy composition can be processed to prepare a biodegradable iron-based alloy material in powder form and can also be used as a starting material for various additional processing, to further fabricate biodegradable medical implants with different specific structures. The details are described below.

According to some embodiments of the present disclosure, a biodegradable medical implant is provided. In some embodiments, the biodegradable medical implant can be used as a filling of periodontal tissue (alveolar bone), bone or connective tissue (such as skin) in dental implantation, orthopedic surgery, or plastic surgery. In some embodiments, the biodegradable medical implants include dental implants, orthopedic implants, implants for cardiology and/or implants for plastic surgery.

According to some embodiments of the present disclosure, a method of manufacturing a biodegradable medical implant is provided. The method of manufacturing the biodegradable medical implant includes providing an aforementioned biodegradable iron-based alloy composition and performing an additional process on the biodegradable iron-based alloy composition to form the biodegradable medical implant. The additional process may include an additive manufacturing (AM) process.

In some embodiments, an additive manufacturing (AM) process is implemented by a metal melting process. For example, an energy beam (comprising the power sources, such as laser beam, electron beam, arc, plasma, electromagnetic conduction or the combination thereof) is directed to smelt the biodegradable iron-based alloy composition of the embodiment by way of sintering, melting and solidification or a combination thereof. The sintering process can be a selective laser sintering (SLS) process or a direct metal laser sintering (DMLS) process. The melting process can be a selective laser melting (SLM) process or an electron beam melting (EBM) process. Compared to a typical smelting and hot-pressing process, the laser sintering process or the laser melting process has fast cooling rate (e.g., >10E4 K/sec), thereby forming smaller grains and fabricating a biodegradable medical implant with high mechanical strength.

In some embodiments, the additive manufacturing (AM) process may include steps of providing a biodegradable iron-based alloy composition on a carrier and performing a sintering/melting process on the biodegradable iron-based alloy composition. For example, a focused energy beam can be provided for sintering/melting the biodegradable iron-based alloy composition along a predetermined scan path. Then, the sintered/melted biodegradable iron-based alloy composition is cured to form at least one lamination layer on the carrier. Next, these steps are repeated to form several lamination layers in a stack. Those stacked lamination layers form a block with a three-dimensional structure. In the sintering/melting process according to some embodiments, a laser beam with a power in a range of 200 watts (W) to 340 watts (W) can be used for sintering/melting the powder particles of the biodegradable iron-based alloy composition at a scanning speed in a range of 1500 millimeters per second (mm/s) to 4500 mm/s. In some embodiments, the step of curing the sintered/melted biodegradable iron-based alloy composition may include performing an annealing treatment to the sintered/melted biodegradable iron-based alloy composition in an air atmosphere.

In some embodiments, the biodegradable iron-based alloy compositions can be made into spherical powders using a gas atomization powder manufacturing equipment, and then the spherical powders are sieved using an air-flow powder classifier to separate powders having particle sizes of about 10 μm to 60 μm. Powders of different compositions are mixed using a powder mixer, and those powders are used as a starting material for sintering/melting in the additive manufacturing (AM) process. Thus, several lamination layers are fabricated in a stack to form a block with a three-dimensional structure.

Moreover, the block with a three-dimensional structure formed by stacked lamination layers can be further processed to form different products, such as forming the biodegradable medical implant used in dental implantation, orthopedic surgery, or plastic surgery.

In some embodiments, a predetermined shape of the biodegradable medical implant can be obtained by adjusting the scan path in each sintering/melting process for forming each of the lamination layers of the biodegradable medical implant. As a result, the cross-sectional profile of each lamination layer can be altered due to varied scan paths, and the 3D structure of the biodegradable medical implant that is formed by stacking the lamination layers can thus have the predetermined shape.

According to some embodiments of the present disclosure, a biodegradable medical implant fabricated by an additive manufacturing (AM) process and made of a biodegradable iron-based alloy composition has a tensile strength up to about 1200 MPa. This tensile strength is about 2.4 times the tensile strength of pure iron.

Embodiments are further described below. Several compositions and test results of the characteristics of biodegradable iron-based alloy compositions in some embodiments are provided below, for illustrating the biodegradable iron-based alloy compositions and medical implant as made. However, the following embodiments are provided for exemplification, not for limitation on the present disclosure.

Table 1 lists elemental compositions and weight ratios of the iron-based alloy compositions. Table 2 lists test results of medical implants as fabricated. Examples 1-3 and comparative examples 1-2 are samples of the medical implants fabricated by an additive manufacturing (AM) process. The composition of Comparative Example 1 is substantially pure iron, but trace impurities difficult to remove are also listed in Table 2. The compositions and contents of the trace impurities are measured using X-ray fluorescence analysis (XRF).

The MTT assay has been used to assess cell viability. MTT (%) in Table 2 represents a result of cytotoxicity. A cytotoxicity test is conducted based on the International Organization for Standardization (ISO) 10993-5. In the MTT assay, 3-[4,5-dimethylthiazole-2-yl]-2,5-diphenyltetrazolium bromide (MTT) is reduced by cellular enzymes to the blue product formazan. The reduction of MTT is primarily by the mitochondrial dehydrogenases. The mitochondrial dehydrogenases of viable cells cleave the tetrazolium ring of the MTT of viable cells, yielding blue precipitate. The more living cells there are, the higher the enzyme activity and the absorbance value. Therefore, the absorbance value (such as optical density (O.D.) value) can be used to calculate the percentage of cell viability. In other words, cell viability can be evaluated through the absorbance values. The higher the absorbance values, the greater the percentage of cell viability. If the cell survival ratio is lower than 70%, it is determined that the composition in the test is cytotoxic.

A testing method and evaluation of the biodegradation rate in Table 2 are described below. The samples are measured according to ASTM G102-89. According to the experiments of degradation rate, the degradation rate (mm/year) can be obtained by measuring a corrosion current density (A/cm²) of the testing samples. First, the sample is immersed in simulated body fluid (SBF), and the experimental environment is maintained at 37° C. through a constant temperature water tank. Next, the changes in potential values or current values during experimentations are recorded by the potentiostatic method or the potentiostatic polarization method. The corrosion current is calculated and obtained by Tafel extrapolation method. According to the Tafel extrapolation method, a linear region near 50 mV of corrosion potential is obtained, which is also called a Tafel straight line region. The tangent lines of the cathode and anode curves in the Tafel straight line regions intersect at a horizontal axis. The intersection of the two tangent lines and the horizontal axis is the corrosion current (Icorr), representing the corrosion rate.

TABLE 1

| | | | | | Com- |
|---|---|---|---|---|---|
| | | | | Comparative | parative |
| | Example 1 | Example 2 | Example 3 | Example 1 | Example 2 |
| Fe (wt %) | 98.513 | 98.313 | 98.245 | 99.80 | 99.72 |
| Mn (wt %) | 0.586 | 0.358 | 0.456 | 0.12 | 0.118 |
| Zn (wt %) | 0.152 | 0.482 | 0.853 | N/A | 0.5 |
| Cr (wt %) | 0.207 | 0.126 | 0.116 | 0.04 | 0.036 |
| Cu (wt %) | 0.116 | 0.105 | 0.139 | N/A | 0.038 |
| Mo (wt %) | 0.082 | 0.130 | 0.063 | N/A | 0.05 |
| Si (wt %) | 0.105 | 0.095 | 0.071 | 0.01 | 0.007 |
| C (wt %) | 0.112 | 0.087 | 0.020 | 0.030 | 0.025 |
| Ni (wt %) | 0.070 | 0.093 | N/A | N/A | 0.024 |
| Al (wt %) | N/A | 0.083 | N/A | N/A | 0.019 |
| Zr (wt %) | 0.026 | 0.052 | N/A | N/A | N/A |
| P (wt %) | N/A | 0.043 | 0.038 | N/A | 0.008 |
| Co (wt %) | 0.032 | 0.032 | N/A | N/A | N/A |

TABLE 2

| | | | | |
|---|---|---|---|---|
| | | | Comparative | Comparative |
| Example 1 | Example 2 | Example 3 | Example 1 | Example 2 |

MTT(%)

| | | | | | |
|---|---|---|---|---|---|
| Threaded Bone Nail | 85.8 | 104.22 | 106.29 | 106.29 | 78.05 |
| Porous threaded Bone | 91.79 | 108.17 | 110.03 | 110.03 | 71.71 |
| Nail 1 | | | | | |
| Porous threaded Bone | 125.08 | 103.85 | 106.05 | 106.05 | 76.34 |
| Nail 2 | | | | | |

Biodegradation Rate

| | | | | | |
|---|---|---|---|---|---|
| Corrosion Voltage | −0.694 | −0.695 | −0.679 | −0.704 | −0.657 |
| (V) | | | | | |
| Corrosion Current | 29.18 | 33.59 | 38.13 | 9.05 | 21.52 |
| Density (μA/cm²) | | | | | |
| Corrosion Rate | 0.338 | 0.389 | 0.442 | 0.105 | 0.249 |
| (mm/year) | | | | | |

Mechanical Strength

| | | | | | |
|---|---|---|---|---|---|
| Ultimate Tensile | 1210 | 1200 | 1080 | 495 | 568 |
| Strength (UTS) (MPa) | | | | | |
| Elongation (%) | 12.9 | 14.2 | 13.7 | 17 | 12 |

The results shown in Table 2 indicate that the cell survival ratios of three different bone nails provided in Comparative Example 1 are 106.29%, 110.03% and 106.05%, which show no cytotoxicity. However, the bone nails in Comparative Example 1 have excessively low degradation rates and extremely low mechanical strengths. Therefore, if the bone nails in Comparative Example 1 are used as medical implants, it not only has the risk of breaking in the body, but also is difficult to degrade in the body.

In addition, the results shown in Table 2 indicate that the cell survival ratios of three different bone nails provided in comparative Example 2 are 78%, 72% and 76%, respectively. That is, the biocompatibilities of the bone nails in comparative Example 2 are not as good as the biocompatibilities of the bone nails in examples 1-3.

The results shown in Table 2 indicate that the cell survival ratios of different bone nails fabricated by the biodegradable iron-based alloy compositions in Example 1 are 85.8%, 91.79% and 125.08%. The cell survival ratios of different bone nails fabricated by the biodegradable iron-based alloy compositions in Example 2 are 104.22%, 108.17% and 103.85%. The cell survival ratios of different bone nails fabricated by the biodegradable iron-based alloy compositions in Example 3 are 106.29%, 110.03% and 106.05%. Therefore, the bone nails fabricated by the biodegradable iron-based alloy compositions in Examples 1-3 have no cytotoxicity and are suitable for being the biodegradable medical implants. Also, the bone nails fabricated by the biodegradable iron-based alloy compositions in Examples 1-3 have high tensile strength of about 1080 MPa to 1210 MPa and good elongation (strain) of about 12.9% to 13.5%. Taking the bone nail of Example 2 as an example, the tensile strength (i.e. 1210 MPa) of the bone nail of Example 2 is significantly increased by about 2.4 times of the tensile strength (i.e. 495 MPa) of the bone nail of Comparative Example 1 (substantially pure iron). Thus, the medical implant made of the biodegradable iron-based alloy composition of the embodiments has good biocompatibility, high degradation rate and excellent mechanical strength.

Also, the test results of Examples 1-3 shows that the mechanical strength and the degradation rate of the biodegradable iron-based alloy composition can be adjusted by fine-tuning the weight ratio of the elements in the additional material of the composition. For example, the degradation rate of the biodegradable iron-based alloy composition can be adjusted in a range of about 0.1 mm/year to 1.0 mm/year. Thus, the biodegradable iron-based alloy composition of the embodiments can be adapted to a variety of different medical needs, provides high flexibility in uses and diversity in application.

Moreover, according to some embodiments of the present disclosure, Also, the selective laser melting (SLM) samples of the pure iron and the iron-based alloy compositions of the embodiments are formed, and solid electrolyte interphase (SEI) morphologies of the samples are observed by scanning electron microscope (SEM) after etching. According to the observation results, the SLM sample of the pure iron has an average particle size of greater than 50 μm, such as about 50 μm. The SLM samples of the iron-based alloy compositions have an average particle size of greater than about 5 μm, such as about 10 μm. Compared to a sample formed by the typical smelting and hot-pressing process, which has an average particle size of about 500 μm, the SLM samples have much smaller grains since the laser melting has fast cooling rate. The average particle size of the SLM samples of the iron-based alloy compositions of the embodiments have further decreased to about 5 μm to 10 μm, which is smaller than the average particle size of the SLM sample of the pure iron. Also, according to the observation results, it can be observed that the small grains are uniformly distributed, and the carbide particles are formed at the grain boundaries of the alloy structures after processing the biodegradable iron-based alloy compositions of the embodiments. The medical implant made of the biodegradable iron-based alloy compositions containing the carbide particles would have high hardness and high mechanical strength.

Additionally, several implantation experiments are conducted, wherein the pins made of the biodegradable iron-based alloy compositions of the embodiments are implanted into the femoral mid-diaphyseal region of Sprague-Dawley (SD) rats. In the implantation experiments, the pins are implanted in SD rats for 180 days, and the implanted area of SD rats are checked constantly during the implantation period. After 180 days of implantation, the pins are removed from the SD rats and measured for the mass loss. According to the experimental results of the implantation, there are non-irritant for local tissue response, and the sizes of the pins are clearly reduced. Considerable mass losses of the pins made of the biodegradable iron-based alloy compositions of the embodiments can be obtained. Although the mechanism of in vivo biodegradation is complicated, in vivo corrosion rate can be represented by in vitro corrosion rates to some extent. The in vitro corrosion rate can be obtained through the mass loss of the pin (i.e. by measuring the weights of the pin before and after 180-days implantation). The corrosion rate over an extended period of time can be calculated from the following equation:

Corrosion rate (mm/year)=8.76×10⁴×ΔW/(A×t×ρ)

ΔW: weight loss;

A; original surface area (exposed area);

t: exposure time; and

ρ: standard density of the material.

One of the implantation experiments and related results are provided herein for illustration. In one example, a pin (2 mm diameter, 6 mm length) made of a biodegradable iron-based alloy composition in Example 1 is implanted into the femoral mid-diaphyseal region of SD rats for 180 days. The weight of the pin before implantation is 0.1510±0.0036 g. The implanted area of SD rats are checked constantly during the implantation period, and there are considered as non-irritant for local tissue response as compared to the negative control since the evaluation score is less than 0 (<0), and no specific adverse findings during 180-days observation and in histopathology data. After 180 days of implantation, the pins are removed from the SD rats, and the weight of the pin after implantation is decreased to 0.0530±0.0074 g, wherein the size of the pin is clearly reduced (e.g., the length of the pin after implantation is about 2 mm). The in vitro corrosion rate can be obtained by measuring the weights of the pin before and after 180-days implantation), and calculated by using the equation above. In this example, the corrosion rate of the pin over 180 days is, but not limited to, 0.385 mm/year to 0.479 mm/year. Therefore, the iron-based alloy compositions in some embodiments of the present disclosure are biodegradable and have several advantages, such as non-irritant for local tissue response in the implantation area, excellent biocompatibility, fast degradation rate and high mechanical strength.

According to the aforementioned descriptions, the embodiments of the present disclosure provide a biodegradable iron-based alloy composition. The biodegradable iron-based alloy composition includes at least 98 wt % of iron (Fe) and 2 wt % or less of an additional material. According to some embodiments of the present disclosure, a biodegradable medical implant fabricated by an additive manufacturing (AM) process and made of a biodegradable iron-based alloy composition has significantly improved mechanical strength and degradation rate, and also has no cytotoxicity (i.e. high biocompatibility). Therefore, the biodegradable medical implant made of the embodied biodegradable iron-based alloy composition can help tissues heal in the body without causing inflammation in surrounding tissues, and also can be gradually corroded and degraded until they are completely absorbed and/or metabolized in the body. Accordingly, this can also solve the current problems of non-degradable medical materials requiring the second surgery to remove and a foreign body sensation. Moreover, according to the elemental compositions and weight ratios of the biodegradable iron-based alloy compositions of the embodiments not only solve the problems of insufficient mechanical strength of the polymer-based implants and the Mg-based implants, but also improve the design flexibility of porous implants.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

What is claimed is:

1. A biodegradable iron-based alloy composition, comprising:
at least 98 wt % of iron (Fe); and
2 wt % or less of an additional material, wherein the additional material comprises 0.1 wt %-0.8 wt % of manganese (Mn), 0.01 wt %-0.15 wt % of molybdenum (Mo), 0.1 wt %-0.3 wt % of chromium (Cr), 0.02 wt %-0.15 wt % of carbon (C), and 0.01 wt %-0.15 wt % of silicon (Si).

2. The biodegradable iron-based alloy composition as claimed in claim 1, wherein the additional material further includes 0.01 wt %-0.2 wt % of copper (Cu).

3. The biodegradable iron-based alloy composition as claimed in claim 1, wherein the additional material further includes 0.1 wt %-1.0 wt % of zinc (Zn).

4. The biodegradable iron-based alloy composition as claimed in claim 1, wherein the additional material further includes 0.01 wt %-0.1 wt % of phosphorum (P).

5. The biodegradable iron-based alloy composition as claimed in claim 1, wherein the additional material further includes 0.01 wt %-0.1 wt % of cobalt (Co).

6. The biodegradable iron-based alloy composition as claimed in claim 1, wherein the additional material further includes 0.3 wt %-0.6 wt % of manganese (Mn) and 0.06 wt %-0.12 wt % of silicon (Si).

7. The biodegradable iron-based alloy composition as claimed in claim 1, wherein the additional material further includes 0.02 wt %-0.12 wt % of nickel (Ni).

8. The biodegradable iron-based alloy composition as claimed in claim 1, wherein the additional material further includes 0.01 wt %-0.1 wt % of aluminum (Al).

9. A biodegradable medical implant, comprising:
a biodegradable iron-based alloy composition, comprising:
at least 98 wt % of iron (Fe); and
2 wt % or less of an additional material, wherein the additional material includes 0.1 wt %-0.8 wt % of manganese (Mn), 0.01 wt %-0.15 wt % of molybdenum (Mo), 0.1 wt %-0.3 wt % of chromium (Cr), 0.02 wt %-0.15 wt % of carbon (C), and 0.01 wt %-0.15 wt % of silicon (Si).

10. The biodegradable medical implant as claimed in claim 9, wherein the additional material further includes 0.01 wt %-0.2 wt % of copper (Cu).

11. The biodegradable medical implant as claimed in claim 9, wherein the additional material further includes 0.1 wt %-1.0 wt % of zinc (Zn).

12. The biodegradable medical implant as claimed in claim 9, wherein the additional material further includes 0.01 wt %-0.1 wt % of phosphorum (P).

13. The biodegradable medical implant as claimed in claim 9, wherein the additional material further includes 0.01 wt %-0.1 wt % of cobalt (Co).

14. The biodegradable medical implant as claimed in claim 9, wherein the additional material further includes 0.3 wt %-0.6 wt % of manganese (Mn) and 0.06 wt %-0.12 wt % of silicon (Si).

15. The biodegradable iron-based alloy composition as claimed in claim 9, wherein the additional material further includes 0.02 wt %-0.12 wt % of nickel (Ni).

16. The biodegradable iron-based alloy composition as claimed in claim 9, wherein the additional material further includes 0.01 wt %-0.1 wt % of aluminum (Al).

17. A method of manufacturing a biodegradable medical implant, comprising:
providing a biodegradable iron-based alloy composition, comprising:
at least 98 wt % of iron (Fe); and
2 wt % or less of an additional material, wherein the additional material includes 0.1 wt %-0.8 wt % of manganese (Mn), 0.01 wt %-0.15 wt % of molybdenum (Mo), 0.1 wt %-0.3 wt % of chromium (Cr), 0.02 wt %-0.15 wt % of carbon (C), and 0.01 wt %-0.15 wt % of silicon (Si); and
performing an additive manufacturing process on the biodegradable iron-based alloy composition to form the biodegradable medical implant.
